(19) United States
(12) Patent Application Publication
(10) Pub. No.: US 2017/0216495 A1
(43) Pub. Date: Aug. 3, 2017

(54) COATINGS COMPRISING BIS-(ALPHA-AMINO-DIOL-DIESTER) CONTAINING POLYESTERAMIDE

(71) Applicant: DSM IP Assets B.V., TE Heerlen (NL)

(72) Inventors: Soazig Claude Marie DELAMARRE, BL Maastricht (NL); George MIHOV, BL Maastricht (NL); Astrid FRANKEN, BL Maastricht (NL); Kenneth MESSIER, BL Maastricht (NL)

(21) Appl. No.: 15/493,664

(22) Filed: Apr. 21, 2017

Related U.S. Application Data
(63) Continuation of application No. 14/989,273, filed on Jan. 6, 2016.
Application No. 14/989,273 is a continuation of application No. 13/395,527, filed on Jun. 19, 2012, now abandoned.

(30) Foreign Application Priority Data
Oct. 16, 2009 (EP) 09173349.3

(51) Int. Cl. A61L 31/10; A61F 2/82; A61L 31/14; C09D 177/12; A61L 31/16; A61L 31/06

(52) U.S. Cl. CPC A61L 31/10; A61L 31/16; A61L 31/06; A61L 31/148; C09D 177/12; A61F 2/82; A61L 2420/02; A61L 2420/06; A61L 2300/606; A61L 2300/416; A61L 2300/204; A61L 2300/604; A61F 2210/0004; A61F 2240/001

(57) ABSTRACT

The present invention relates to a coating comprising at least one biodegradable polymer, wherein the polymer comprises at least one or a blend of a poly (ester amide) (PEA) having a chemical formula described by structural formula (II), wherein; R₁ is independently selected from the group consisting of (C₂-C₂₀)alkylene, (C₂-C₂₀)alkenylene, —(R₉—CO—O—R₁₀—O—CO—R₉)—, CH R₁₁—O—CO—R₁₂—COOCR₁₁— and combinations thereof; R₃ and R₄ in a single co-monomer m or p, respectively, are independently selected from the group consisting of hydrogen, (C₁-C₆)alkyl, (C₂-C₆)alkenyl, (C₂-C₆)alkynyl, (C₆-C₁₀)aryl, (C₁C₆)alkyl, —(CH₂)SH, —(CH₂)₂S(CH₃), CH₂OH, —CH(OH)CH₃, —(CH₂)₄NH₃+, ˜(CH₂)₃NHC(═NH₂+)NH₂, —CH₂COOH, (CH₂)COOH, —CH₂—CO—NH₂—CH₂CH₂—CO—NH₂, —CH₂CH₂COOH, CH₃—CH₂—CH(CH₃)—, formula (a), HO-_{P}-Ph-CH₂—, (CH₃)₂—CH—, Ph- NH—, NH—(CH₂)₃—C—, NH—CH═N—CH═C—CH₂—. R₅ or R₆ are independently selected from bicyclic-fragments of 1,4:3,6-dianhydrohexitols or from the group consisting of (C₂-C₂₀)alkylene, (C₂-C₂₀)alkenylene, alkyloxy, oligoethyleneglycol with a Mw ranging from 44 Da up to 700 Da, —CH₂—CH—(CH₂OH)₂, CH₂CH(OH)CH₂ whereby R₅ and R₆ are non identical. R₇ is hydrogen, (C₆-C₁₀) aryl, (C₁C₆) alkyl or a protecting group such as benzyl- or a bioactive agent; R₈ is independently (C₁-C₂₀) alkyl or (C₂-C₂₀)alkenyl; R₉ or R₁₀ are independently selected from C₂-C₁₂ alkylene or C₂-C₁₂ alkenylene and R₁₁ or R₁₂ are independently selected from H, methyl, C₂-C₁₂ alkylene or C₂-C₁₂ alkenylene.

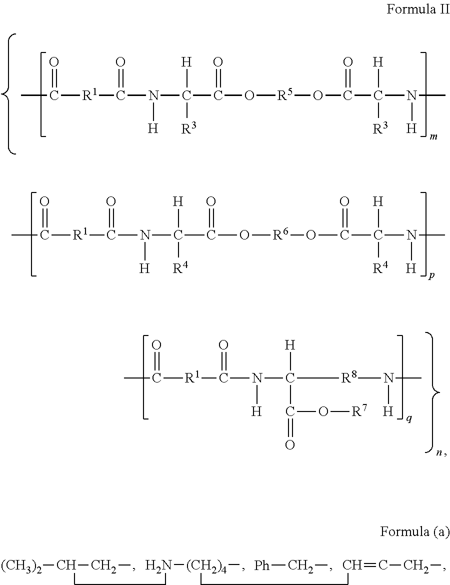

This application is a continuation of application Ser. No. 14/989,273, filed Jan. 6, 2016, pending, which is a continuation of application Ser. No. 13/395,527, filed Jun. 19, 2012, abandoned, which is the U.S. national phase of International Application No. PCT/EP2010/065663 filed 18 Oct. 2010 which designated the U.S. and claims priority to EP 09173349.3 filed 16 Oct. 2009, the entire contents of each of which are hereby incorporated by reference.

The present invention relates to coatings comprising α-amino acid-diol-diester containing polyesteramides (PEA).

α-amino acid-diol-diester based polyesteramides (PEA) are well known in the art and disclosed by G. Tsitlanadze, et al. J. Biomater. Sci. Polym. Edn. (2004) 15:1-24 who showed enzyme-mediated surface degradation and a low inflammation profile (K. DeFife et al. Transcatheter Cardiovascular Therapeutics—TCT 2004 Conference). These properties make the PEA excellent materials for a variety of different medical and pharmaceutical applications. The physical and mechanical properties as well as biodegradable profiles can be adjusted simply by varying three components in the building blocks during their synthesis the alpha-amino acids, the diols and the aliphatic dicarboxylic acids.

Coatings comprising α-amino acid-diol-diester based polyesteramides and the use of these polymers on a medical device such as a stent are disclosed in EP-A-1603485. EP-A-1603485 relates to coatings comprising alpha-amino acid-diol-diester based polyesteramides (PEA) of formula I, further referred to as PEA-I,

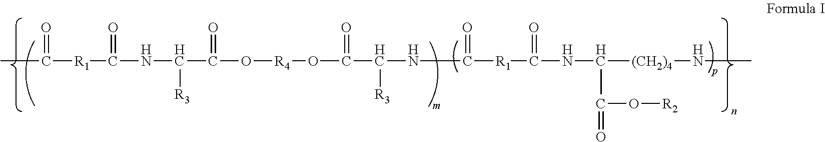

wherein:
m is about 0.1 to about 0.9; p is about 0.9 to about 0.1; n is about 50 to about 150;
each R1 is independently (C1-C₂₀)alkylene; each R₂ is independently hydrogen, or (C₆-C₁₀)aryl(C1-C₆)alkyl;
each R₃ is independently hydrogen, (C1-C₆) alkyl, (C₂-C₆)alkenyl, (C₂-C₆)alkynyl, or (C₆-C₁₀)aryl(C₁-C₆)alkyl; and each R4 is independently (C₂-C₂₀)alkylene.

PEA-I is a copolymer comprising alpha-amino acids, diols and an aliphatic dicarboxylic acids, which is copolymerized with an aliphatic dicarboxylic acid and lysine. A bioactive agent may covalently bound to the carboxylic group of the lysine part.

As shown in the examples coatings of PEA-I have been tested together with a covalently bound bioactive agent such as 4-amine TEMPO on stents. The polymer was shown to be a safe form of a bioabsorbable polymer. The application is however silent about the release of the bioactive agent 4-amine-TEMPO out of the PEA-I coating.

There is however a need for coatings, comprising PEA's and bioactive agents, from which the release is uniform and from which the release rate of the bioactive agents can be tailored.

The object of the present invention is therefore to provide a coating comprising PEA and a bioactive agent from which the release and release rate can be easily tuned.

A further object of the present invention is to provide a coating comprising PEA and a bioactive agent from which the release pattern is uniform, not showing a burst release in the first 24 hours.

Another object of the present invention is to provide a coating comprising PEA and a bioactive agent from which a release pattern can be shown on a longer term.

The object of the present invention is achieved by providing a coating comprising at least one biodegradable polymer and a dispersed bioactive agent wherein the polymer comprises at least one or a blend of a poly (ester amide) (PEA) having a chemical formula described by structural formula (II),

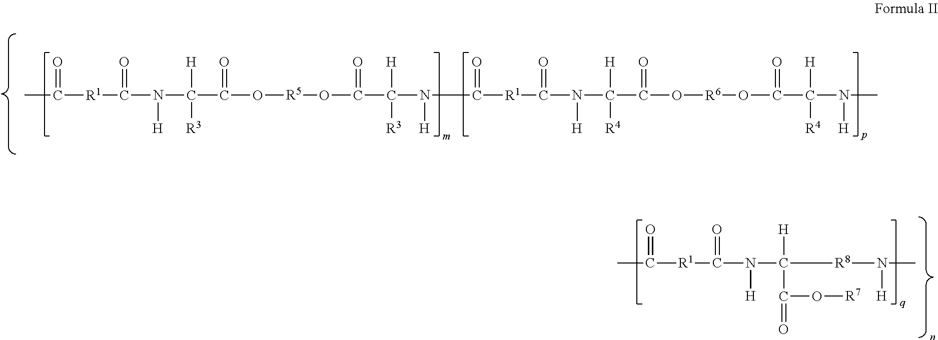

wherein
m is about 0.01 to about 0.99; p is about 0.99 to about 0.01; and q is about 0.99 to 0.01; and wherein n is about 5 to about 100; and wherein
R₁ is independently selected from the group consisting of (C₂-C₂₀)alkylene, (C₂-C₂₀)alkenylene, —(R₉—CO—O—R₁₀—O—CO—R₉)—, —CHR₁₁—O—CO—R₁₂—COOCR₁₁— and combinations thereof;
R₃ and R₄ in a single co-monomer m or p, respectively, are independently selected from the group consisting of hydrogen, (C₁-C₆)alkyl, (C₂-C₆)alkenyl, (C₂-C₆)alkynyl, (C₆-C₁₀)aryl, (C₁-C₆)alkyl, —(CH₂)SH, —(CH₂)₂S(CH₃), —CH₂OH, —CH(OH)CH₃, —(CH₂)₄NH₃+, —(CH₂)₃NHC(═NH₂+)NH₂, —CH₂COOH, —(CH₂)COOH, —CH₂—CO—NH₂, —CH₂CH₂—CO—NH₂, —CH₂CH₂COOH, CH₃—CH₂—CH(CH₃)—, (CH₃)₂—CH—CH₂—, H₂N—(CH₂)₄—, Ph-CH₂—, CH═C—CH₂—, HO-p-Ph-CH₂—, (CH₃)₂—CH—, Ph-NH—,

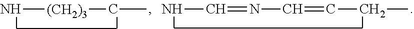

R₅ or R₆ are independently selected from bicyclic-fragments of 1,4:3,6-dianhydrohexitols or from the group consisting of (C₂-C₂₀)alkylene, (C₂-C₂₀)alkenylene, alkyloxy, oligoethyleneglycol with a Mw ranging from 44 Da up to 700 Da, —CH₂—CH—(CH₂OH)₂, CH₂CH(OH)CH₂ and whereby R₅ and Re are non identical and whereby at least one of R5 or R6 is a bicyclic-fragment of 1,4:3,6-dianhydrohexitols,
R₇ is hydrogen, (C₆-C₁₀) aryl, (C₁-C₆) alkyl or a protecting group such as benzyl- or a bioactive agent;
R₈ is independently (C₁-C₂₀) alkyl or (C₂-C₂₀)alkenyl;
R₉ or R₁₀ are independently selected from C₂-C₁₂ alkylene or C₂-C₁₂ alkenylene.
R₁₁ or R₁₂ are independently selected from H, methyl, C₂-C₁₂ alkylene or C₂-C₁₂ alkenylene
   suitable for coating an implantable device.

### BRIEF DESCRIPTION OF THE DRAWINGS

FIG. 1 depicts the result of an average of 4 measurements for PEA II and PEA III coatings.

### DETAILED DESCRIPTION OF THE INVENTION

The coating of the present invention is based on a polyesteramide comprising an extra block p compared to the above disclosed prior art PEA's of Formula I. This kind of PEA blockcopolymers has been found to provide excellent properties in terms of release of a bioactive agent and provide excellent properties in tuning the release of bioactive agents by adjusting the amount of m, p, q blocks. Moreover it has been found that this polymer is holding the drug without being covalently bound so that an initial burst release can be avoided. The coating moreover ensures a uniform release of the bioactive agents for at least 20 days.

The PEA polymers as such are known in the art and disclosed in US2008/0299174. US2008/0299174 discloses the PEA polymers based on bis-(a-amino acid)-diol-diesters containing two bis-(a-amino acid)-based building blocks and shows the polymers to provide a significant improvement in mechanical properties. Incorporation of at least two linear saturated or unsaturated aliphatic diol residues into the two bis-(a amino acid)-based (e.g. bis-(a-amino acid)-diol-diester co-monomers of a PEA), increases the elongation properties of the resulting polymer. The PEA co-polymers seem to be suitable for certain applications requiring a combination of hydrophobicity, relatively high glass transition temperature (Tg), and properties of variable elongation or flexibility. Furthermore methods are disclosed for fixing a fixation device made of the PEA's into the internal body site. The device biodegrades to create substantially biocompatible breakdown products while fixing the internal body site. Also biocompatible surgical devices fabricated using the PEA compositions are disclosed. The disclosure is however silent about coatings based on the PEA's for the release of bioactive agents.

Accordingly, in a preferred embodiment, the invention provides coatings comprising PEA co-polymer compositions having a chemical structure described by general structural formula (II): wherein
m is about 0.01 to about 0.99; p is about 0.99 to about 0.01; and q is about 0.99 to 0.01; and wherein n is about 5 to about 100; and
R₁ is independently selected from the group consisting of (C₂-C₁₀)alkylene such as (CH₂)₄ or (CH₂)₈ or (C₂-C₂₀)alkenylene, and combinations thereof;
R₃s and R₄s in a single co-monomer m or p, respectively, are independently selected from the group consisting of hydrogen, (C₁-C₆)alkyl, (C₂-C₆)alkenyl, (C₂-C₆)alkynyl, (C₆-C₁₀)aryl (C₁-C₆)alkyl and —(CH₂)₂S(CH₃);
R₅ is selected from bicyclic-fragments of 1,4:3,6-dianhydrohexitols of structural formula (III);

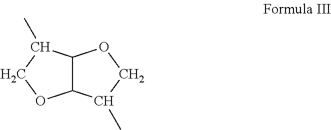

R₆ is selected from the group consisting of (C₂-C₂₀)alkylene such as cyclohexanediol, (C₂-C₂₀)alkenylene or alkyloxy;
R₇ is benzyl and
R₈ is independently (C₃-C₆) alkyl or (C₃-C₆) alkenyl.

As used herein, the term “alkyl”, refers to a straight or branched chain hydrocarbon group including methyl, ethyl, n-propyl, isopropyl, n-butyl, isobutyl, tert-butyl, n-hexyl, and the like.

As used herein, “alkenyl” or “alkenylene”, refers to structural formulas herein to mean a divalent branched or unbranched hydrocarbon chain containing at least one unsaturated bond in the main chain or in a side chain.

As used herein, “alkynyl”, refers to straight or branched chain hydrocarbon groups having at least one carbon-carbon triple bond.

The term “aryl” is used with reference to structural formulas herein to denote a phenyl radical or an ortho-fused bicyclic carbocyclic radical having about nine to ten ring atoms in which at least one ring is aromatic. Examples of aryl include, but are not limited to, phenyl, naphthyl, and nitrophenyl.

At least one of the alpha-amino acids used in the co-polymers is a natural alpha-amino acid. For example, when the R₃s or R₄s are CH₂Ph, the natural alpha-amino acid used in synthesis is L-phenylalanine. In alternatives wherein the R₃s or R₄s are CH₂—CH(CH₃)₂, the co-polymer contains the natural amino acid, leucine. By independently varying the R₃s and R₄s within variations of the two co-monomers as described herein, other natural alpha-amino acids can also be used, e.g., glycine (when the R₃s or R₄s are H), alanine (when the R₃s or R₄s are CH₃), valine (when the R₃s or R₄s are CH(CH₃)₂), isoleucine (when the R₃s or R₄s are CH(CH₃)—CH₂—CH₃), phenylalanine (when the R₃s or R₄s are CH₂—C₆H₅), lysine (when the R₃s or R₄s (CH₂)₄—NH₂); or methionine (when the R₃s or R₄s are —(CH₂)₂S(CH₃), and mixtures thereof.

The PEA co-polymers preferably have an average number molecular weight (Mn) ranging from 15,000 to 200,000 Daltons. The PEA co-polymers described herein can be fabricated in a variety of molecular weights and a variety of relative proportions of the two bis-(alpha amino acid)-containing units and optional Lysine-based monomer of the co-polymer. The appropriate molecular weight for a particular use is readily determined by one of skill in the art. A suitable Mn will be in the order of about 15,000 to about 100,000 Daltons, for example from about 30,000 to about 80,000 or from about 35,000 to about 75,000. Mn is measured via GPC in THF with polystyrene as standard.

Further properties and methods of manufacturing the PEA's are disclosed in US2008/0299174 which is herein incorporated by reference.

It has been found that the nature of the PEA polymer plays an important role in defining the surface properties of a coating. For example, coating integrity depends largely on the nature of the polymer forming the coating. A polymer providing a very low Tg, will result in an amorphous coating material which has unacceptable rheological behavior upon mechanical perturbation such as crimping, expansion, etc. On the other hand, a polymer providing a high Tg or highly crystalline coating material will become brittle in the high strain areas when for example coated on a medical device. The PEA's used in the coating of the present invention comprise the incorporation of a bicyclic-fragment of 1,4:3,6-dianhydrohexitol as the diol residue in at least one of the two bis (a-amino acid)-based building blocks which confers a (Tg) above body temperature. By further varying the other building blocks in the PEA Tg can be adjusted further. Preferably the Tg of the PEA ranges from about 40 to about 65. Tg is measured by DSC.

Surprisingly it has been found that the release time can be easily tailored by varying the building blocks of the polymer and by varying the amount of the m, p, q blocks in the PEA copolymer. Moreover the polymer/drug ratio plays an important role in the tuning of the release. Preferably the polymer/drug ratio is 60/40 (w %/w %), more preferably the polymer/drug ratio is 70/30 (w %/w %). Still more preferably the polymer/drug ratio is 75/25 (w %/w %). The polymer/drug ratio is however dependent on the nature of the bioactive agent, the application and on the desired coating thickness.

The coating according to the present invention is preferably a single layer coating. It is even more surprising that the release can be tuned from a single layer coating as the prior art coatings normally require more layers to tune the release of the bioactive agents or to adhere the drug containing PEA layer to the surface of the implantable device.

The coating according to the present invention preferably has a thickness from about 1 μm to 100 μm. More preferably the coating has a thickness of about 2-75 μm, still more preferably a thickness of about 2-50 μm, most preferably a thickness of about 2-15 μm. The coating will lose 100% of its mass within about 12 months.

The bioactive agent which is dispersed with the PEA can be any agent which is a therapeutic, prophylactic, or diagnostic agent. These agents can have antiproliferative or anti-inflammatory properties or can have other properties such as antineoplastic, antiplatelet, anti-coagulant, anti-fibrin, antithrombotic, antimitotic, antibiotic, antiallergic, or antioxidant properties. Moreover, these agents can be cystostatic agents, agents that promote the healing of the endothelium, or agents that promote the attachment, migration and proliferation of endothelial cells while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules, which bind to complementary DNA to inhibit transcription, and ribozymes. Some other examples of bioactive agents include antibodies, receptor ligands, enzymes, adhesion peptides, blood clotting factors, inhibitors or clot dissolving agents, such as streptokinase and tissue plasminogen activator, antigens for immunization, hormones and growth factors, oligonucleotides such as antisense oligonucleotides and ribozymes and retroviral vectors for use in gene therapy. Examples of antiproliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include ABT-578, 40-0-(3-hydroxy)propyl-rapamycin, 40-O-[2-(2-hydroxy)ethoxy]ethyl-rapamycin, and 40-0-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. ADRIAMYCIN® from Pharmacia AND Upjohn, Peapack N.J.), and mitomycin (e.g. MUTAMYCIN® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein Hb/nia platelet membrane receptor antagonist antibody, recombinant hirudin, thrombin inhibitors such as ANGIOMAX® (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck and Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), super oxide dismutases, super oxide dismutase mimetic, 4-amino-2,2,6,6-tetramethylpiperidine-I-oxyl (4-amino-TEMPO), estradiol, anticancer agents, dietary supplements such as various vitamins, and a combination thereof. Examples of anti-inflammatory agents including steroidal and nonsteroidal anti-inflammatory agents include biolimus, tacrolimus, dexamethasone, clobetasol, corticosteroids or combinations thereof. Examples of such cytostatic substances include angiopeptin, angiotensin converting enzyme inhibitors such as captopril (e.g. CAPOTEN® and CAPOZIDE® from Bristol-Myers Squibb Co., Stamford, Conn.), cilazapril or lisinopril (e.g. PRINIVIL® and PRINZIDE® from Merck and Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, and genetically engineered epithelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting.

The coating according to the present invention may comprise a further bioactive agent which means a second or even third bioactive agent. That further bioactive agent can be chosen from the above mentioned bioactive agents. Preferably the further bioactive agent is chosen from growth factors (VEGF, FGF, MCP-1, PIGF, antibiotics, anti-inflammatory compounds, antithrombogenic compounds, anti-claudication drugs, anti-arrhythmic drugs, anti-atherosclerotic drugs, antihistamines, cancer drugs, vascular drugs, ophthalmic drugs, amino acids, vitamins, hormones, neurotransmitters, neurohormones, enzymes, imaging agents, signalling molecules and psychoactive medicaments.

The coating according to the present invention may comprise the dispersed bioactive agent or the further bioactive agent(s) in the form of microparticles, nanoparticles or micelles.

In a further embodiment the coating according to the present invention may be formed of the PEA polymer described herein alone or with one or more other polymers. Representative polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(trimethylene carbonate), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and copolymers of hydroxyl bearing monomers such as HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof. In some embodiments, the coating described herein can exclude any one of the aforementioned polymers.

In a still further embodiment, the coating can further include a biobeneficial material. The biobeneficial material can be polymeric or non-polymeric. The biobeneficial material is preferably substantially non-toxic, non-antigenic and non-immunogenic. A biobeneficial material is one that enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly (ethylene glycol) acrylate (PEGA), PEG methacrylate, 2-methacryloyloxyethylphosphorylcholine (MPC) and <<-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, PolyActive®, or combinations thereof. In some embodiments, the coating can exclude any one of the aforementioned polymers. The term PolyActive® refers to a block copolymer having flexible poly(ethylene glycol) and poly(butylene terephthalate) blocks (PEGTVPBT). PolyActive® is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

The present invention further relates to an implantable device comprising the coating according to the present invention. The implantable device herein can be used to treat, prevent, or ameliorate a medical condition such as atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct.

As used herein, an implantable device may be any suitable medical substrate that can be implanted in a human or veterinary patient. Examples of such medical devices include self-expandable stents, balloon-expandable stents, stent-grafts, grafts (e.g., aortic grafts), heart valve prostheses, cerebrospinal fluid shunts, pacemaker electrodes, catheters, and endocardial leads (e.g., FINELINE and ENDOTAK, available from Guidant Corporation, Santa Clara, Calif.), anastomotic devices and connectors, orthopedic implants such as screws, spinal implants, and electro-stimulatory devices. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-indium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35 percent cobalt, 35 percent nickel, 20 percent chromium, and 10 percent molybdenum. “MP20N” consists of 50 percent cobalt, 20 percent nickel, 20 percent chromium, and 10 percent molybdenum. Devices made from bioabsorbable (e.g., bioabsorbable stent) or biostable polymers could also be used with the embodiments of the present invention.

Preferably, the implantable device is a stent. The stent described herein is useful for a variety of medical procedures, including, by way of example, treatment of obstructions caused by tumors in bile ducts, esophagus, trachea/bronchi and other biological passageways. A stent having the above-described coating is particularly useful for treating diseased regions of blood vessels caused by lipid deposition, monocyte or macrophage infiltration, or dysfunctional endothelium or a combination thereof, or occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, carotid and coronary arteries.

The polymers described herein can be coated onto the surface of the implantable device in many ways, such as dip-coating, spray-coating, ionic deposition, and the like, as is well known in the art. Preferably the coating of the present invention is spray coated on an implantable device.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutically effective dosages can be determined empirically, for example by infusing vessels from suitable animal model systems and using immuno-histochemical, fluorescent or electron microscopy methods to detect the agent and its effects, or by conducting suitable in vitro studies. Standard pharmacological test procedures to determine dosages are understood by those of ordinary skill in the art.

As used herein, “biodegradable” means that at least the polymer is capable of being broken down into innocuous and bioactive products in the normal functioning of the body. The biodegradable polymers have hydrolysable ester linkages which provide the biodegradability, and are typically chain terminated with carboxyl groups.

As used herein, the terms “alpha-amino acid” mean a chemical compound containing an amino group, a carboxyl group and R3 or R4 groups as defined herein. As used herein, the alpha amino acid mean the alpha-amino acid(s) used in synthesis are naturally occurring L-phenylalanine, leucine, glycine, alanine, valine, isoleucine, lysine, or methionine, or a mixture thereof. Additional natural amino acids include lysine and ornithine.

As used herein the term “bioactive agent” means an agent, for example as described herein, having a therapeutic, healing or palliative effect in mammals, including humans. A bioactive agent as disclosed herein is not incorporated into the co-polymer backbone, but is dispersed within the PEA co-polymer. In one embodiment, at least two different bioactive agents are dispersed in co-polymer. As used herein, the term “dispersed” as used to refer to bioactive agents, means the bioactive agents are intermixed, dissolved, or homogenized with the PEA co-polymer.

The present invention will now be described in detail with reference to the following non limiting examples which are by way of illustration only.

### EXAMPLES

### Materials and Methods.

Phosphate Buffer Saline (PBS) was purchased from Biochrom AG.

Rapamycin was used as received from Cfm Oskar Tropitzsch e.K.

### In Vitro Release Method:

A metal alloy stent is incubated in 2 ml PBS buffer under static conditions at 37° C. The buffer is exchanged after specific time points. The drug (Rapamycin) release is determined by photometric UV-measurement at 278 nm.

### Example 1

A coating formulation is prepared by dissolving Rapamycin and PEA-3Bz polymer (PEA III) of formula IV in an easily evaporating solvent. The coating formulation is spray-coated onto a stent and dried at room temperature. The resulting coating has a ratio of polymer/drug of 60/40 (w %/w %) and a coating thickness of about 5-6 μm.

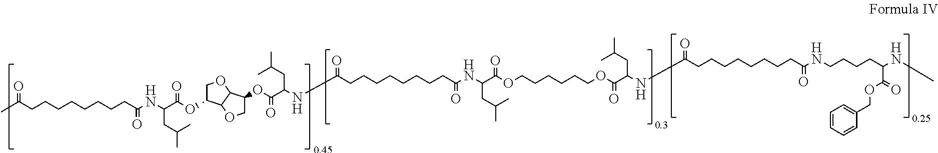

### Example 2

A coating formulation is prepared by dissolving Rapamycin and PEA-2Bz (PEA II) polymer of Formula V in an easily evaporating solvent. The coating formulation is spray-coated onto the stent and dried at room temperature. The resulting coating has a ratio of polymer/drug of 60/40 (w %/w %) and a coating thickness of about 7 μm.

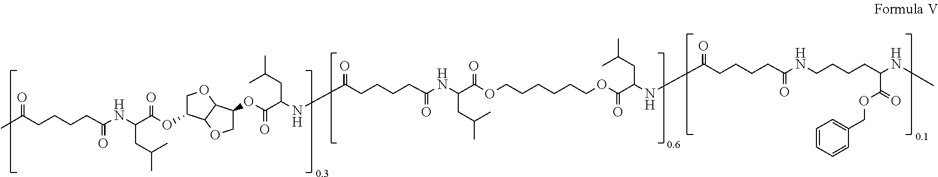

### Results:

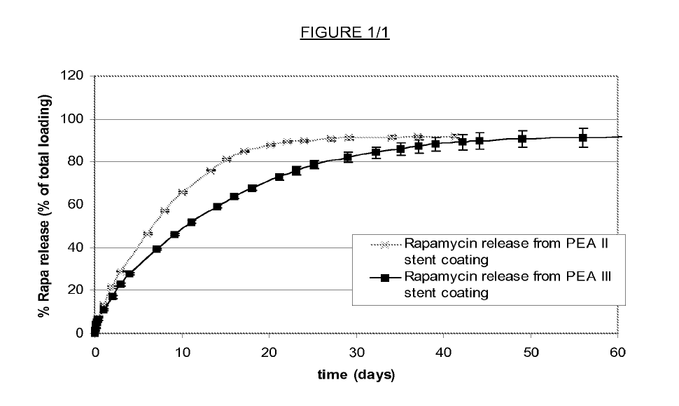

Stent coatings from PEA II and PEA III with Rapamycin prepared under comparable conditions show a faster release in case of PEA II. PEA II coatings could release Rapamycin for about 20 days, while coatings from PEA III could release Rapamycin for about 45 days. These results are shown in FIG. 1. FIG. 1 is the result of an average of 4 measurements for PEA II and PEA III coatings.

1-11. (canceled)

12) A method for coating a stent, comprising the step of spray coating a stent with a composition consisting of:
a) rapamycin;
b) a solvent; and
c) a poly(ester amide) having a chemical structure according to Formula (II): 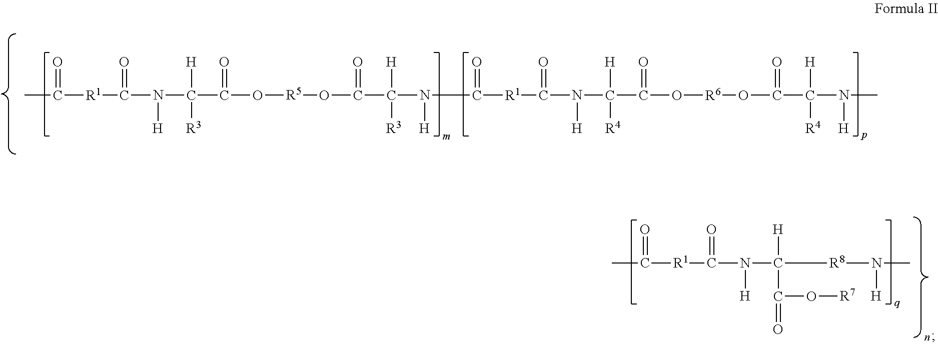
wherein m is about 0.01 to about 0.99;
wherein p is about 0.99 to about 0.01;
wherein q is about 0.99 to 0.01;
wherein n is about 5 to about 100;
wherein R¹ is —(CH₂)₈—;
wherein R³ and R⁴ are —CH₂—CH(CH₃)₂;
wherein R⁵ is 1,4:3,6-dianhydrosorbitol (DAS);
wherein R⁶ is (C₂-C₂₀)alkylene;
wherein R⁷ is a benzyl-protecting group; and
wherein R⁸ is —(CH₂)₄—.

13) The method according to claim 12, wherein the ratio of polymer to drug in the composition is from 60:40 (w %/w %) to 75:25 (w %/w %).

14) The method according to claim 12, wherein the stent is a self-expandable stent or a balloon expandable stent.

15) The method according to claim 12, wherein the stent is made of a bioabsorbable or biostable polymer.

16) The method according to claim 12, wherein the stent is made of a metallic material or an alloy which is at least one selected from the group consisting of stainless steel, cobalt chromium alloy, high nitrogen stainless steel, tantalum, nickel-titanium alloy, platinum-indium alloy, gold, and magnesium.

17) A stent comprising a coating formed according to the method of claim 12.

18) A stent comprising a coating formed according to the method of claim 13.

19) The stent according to claim 17, wherein the coating has thickness of 2-15 μm.

20) The stent according to claim 18, wherein the coating has thickness of 2-15 μm.
